(19) United States
(12) Patent Application Publication
(10) Pub. No.: US 2009/0192355 A1
(43) Pub. Date: Jul. 30, 2009

(54) SCOPE FOR MANAGING DIFFICULT PATHWAYS AND METHOD TO IMPROVE VISIBILITY OF THE SAME

(76) Inventor: Mauricio Mejia, Denver, CO (US)

Correspondence Address: SHERIDAN ROSS PC, 1560 BROADWAY, SUITE 1200, DENVER, CO, 80202, US

(21) Appl. No.: 12/020,862

(22) Filed: Jan. 28, 2008

(51) Int. Cl. A61B 1/267; A61B 1/005; A61B 1/012

(52) U.S. Cl. 600/120; 600/139; 600/146; 600/156

(57) ABSTRACT

A scope adapted for insertion, manipulation and improved visibility in a difficult pathway is disclosed. The scope comprises at least one module for manipulating the scope. The scope may further comprise an illumination source, an image sensor, a power source, and a viewing member for viewing images of a cavity or other anatomical member of a patient. The scope may further comprise alternative pathways for air or fluid flow to exit the distal tip for the purpose of minimizing trauma and improving visibility. In one embodiment the scope is intended to facilitate insertion of an intubating device, which comprises an elongated semi-rigid stylet including first and second ends and at least one inner lumen connected to a module. Additionally, a flexible tip is provided for manipulating one end of the scope and allowing greater flexibility when maneuvering a difficult pathway. A method for navigating a difficult pathway is also disclosed.

### FIELD OF THE INVENTION

The present invention relates generally to medical devices for examining a cavity or orifice of a patient. More specifically, it relates to one of a variety of scopes, such as a scope for orotracheal intubation, which provides a method to obtain an enhanced view of the patient's cavity or orifice, and further provides flexibility to allow direction of a distal tip located on the scope to facilitate insertion and manipulation of the scope.

### BACKGROUND

Many medical procedures require insertion and manipulation of a scope; some scopes used in the medical field include: borescopes, fiberscopes, videoscopes, neurosurgical scopes and intubating scopes. Taking the example of an intubating scope, the procedure often requires insertion of the scope into an endotracheal tube (ETT), which is further inserted into the trachea of a patient. The ETT ensures proper ventilation in the patient, and also allows for the delivery of various gases to the patient, such as an anesthetic or oxygen.

In a typical intubation procedure, the ETT is introduced through the mouth of the patient. Simultaneously, a metal laryngoscope blade (i.e. Miller or MAC Blade) can be used to move the patient's tongue so that the patient's epiglottis and vocal cords can be viewed by the operator. The ETT is then advanced until it is positioned at the proper location in the patient's trachea. Once the ETT is properly positioned, a cuff affixed to the ETT can be inflated to seal the patient's airway passage and allow for the flow of ambient gases. A proper procedure firmly fixes the endotracheal tube in place in the patient's trachea. At this time, the laryngoscope can be withdrawn leaving the ETT in the body.

In some situations, the patient's epiglottis or anatomical features, blood or other secretions, and sometime even debris, may present what is known as a “difficult airway”. In a difficult airway situation, the currently available metal laryngoscope blades can cause trauma to soft tissue, teeth and other areas of the patient due to, in part, the size, rigidity and low versatility of the blade. As a result, some practitioners have begun using flexible scopes which are inserted into the ETT, some of which allow the practitioner to view the airway during insertion of the ETT into the trachea via fiber-optics, to avoid inducing trauma to sensitive features of the airway. The tip of the scope contains an imaging element which communicates images from the distal end of the scope (typically located near the distal end of the ETT) to the proximal end of the scope, and then to a portable monitor or eyepiece. The images displayed on the monitor or eyepiece can be viewed by the operator during insertion of the ETT.

However, prior art devices currently available are ineffective for manipulating soft tissue in the airway and dealing with secretions and other debris to obtain a clear view of the tracheal inlet. If the tip of the scope is covered or obsecured by soft tissue, secretions or other debris, the practitioner will obtain an inaccurate or incomplete image of the trachea, and an effective intubation will likely be delayed. Patients in emergency situations require effective intubations on the practitioner's first intubation attempt.

For example, U.S. Pat. No. 5,817,015 to Adair discloses an endoscope having at least one longitudinal channel formed around its periphery for transmitting fluids or for receiving an operative instrument or carrying light transmitting fibers. However, Adair does not disclose an apparatus capable of injecting gas or fluid other than in a longitudinal direction, and thereby providing freedom to navigate a scope in a tight or difficult passageway.

U.S. Patent Application Publication No. US 2006/0047184 to Banik, et al. discloses an endoscopic imaging system for examining a patient's body cavity including an endoscope having a distal end, a proximal end and a number of lumens therein. One or more distal gas ports are disposed at or adjacent the distal end of the endoscope. Banik et al. also fails to disclose injecting gas or fluid in a non-longitudinal direction, and furthermore does not provide for the symmetrical arrangement of ports as does the current invention.

U.S. Pat. No. 5,685,823 to Ito, et al. discloses an endoscope including a front end having fluid discharge openings, and further having a fluid injection nozzle connected to the fluid discharge opening. The '823 Patent only discloses the injection of fluid through discharge openings located at the front end, which are limited in the direction of flow of fluid or other substance transmitted through the injection nozzles.

U.S. Pat. No. 5,464,008 to Kim discloses a defogger for the objective lens of a laparoscope providing a channel in a longitudinal direction of the laparoscope. Gas from an insufflator is supplied to the channel exteriorly of a body being operated upon. While the channel directs gas across the surface of the objective lens, it does not direct gos or fluid outwardly for clearing an area in front of the lens.

Thus, a need exists for providing an intubating scope that can be used in conjunction with an ETT in difficult airway situations that is effective in dealing with obstructive soft tissue, secretions and other debris, and is easy to use. Furthermore, there is a need for an intubating scope that is flexible and allows for redirection of the distal tip within the difficult airway.

### SUMMARY OF THE INVENTION

These and other needs are addressed by the various embodiments and configurations of the present invention:

It is an object of the present invention to provide a scope for insertion into a cavity or orifice of a patient, such as an intubating scope, which comprises a flexible, controllable tip to allow a practitioner to navigate a difficult pathway while avoiding or minimizing patient trauma.

It is another object of the present invention to provide a scope that provides a clear image of a patient's trachea or other cavity during insertion so as to avoid or minimize trauma to the patent and to facilitate navigation and locate a path for insertion of the scope. The scope includes alternative pathways for air or fluid flow to exit the distal tip for the purpose of minimizing trauma and improving visibility.

According to one embodiment of the present invention, the scope is adapted to be used with an endotracheal tube during orotracheal intubation is provided that includes a module for manipulating the intubating scope. The module includes an illumination source, an image sensor, a power source, and a viewing member. Further, an elongated semi-malleable stylet including first and second ends and at least one inner lumen therein is connected to the module at the first end thereof. Additionally, a first end of a flexible tip is connected to a second end of the stylet. The flexible tip includes first and second ends, at least one inner lumen extending from the first end to the second end, and at least one pathway extending from the first end to the second end and spaced apart from the inner lumen.

When connected, the inner lumen of the stylet is coaxial with the inner lumen of the flexible tip. Furthermore, the intubating scope includes at least one fiber-optic bundle having first and second ends. The first end of the bundle is mounted within the module and the second end of the bundle is mounted within the flexible tip, wherein the bundle includes illumination fibers and/or imaging fibers for allowing viewing of a cavity of a patient via the flexible tip.

According to another embodiment of the present invention, a modified flexible tip is adapted to be used with a scope during insertion and manipulation of the scope is provided. The flexible tip comprises a first end and a second end, and an outer layer connected to an inner layer. The flexible tip further includes a lumen located within the inner layer that extends from the first end to the second end. Moreover, the flexible tip includes at least one tunnel or pathway located within the outer layer and extending from the receiving area to an outlet port at a first location near the second end.

In another embodiment, the flexible tip includes at least one other pathway located within the outer layer. The at least one other pathway extends from the first end of the flexible tip to an outlet port at a second location spaced apart from the first location near the second end.

According to another embodiment of the present invention, the distal tip includes alternative pathways for air or fluid flow to exit the scope for the purpose of minimizing trauma and improving visibility. These pathways manipulate the direction of flow of air and fluid, creating a clean zone in front of the lens by clearing debris, secretions and soft tissue away from the distal tip.

Thus according to one embodiment of the present invention, a flexible tip for use with a scope is disclosed which comprises:

a first end and a second end;

an outer layer and an inner layer extending longitudinally from the first end to the second end;

a lumen located within the inner layer and extending longitudinally from the first end to the second end;

the flexible tip further comprising at least four pathways exterior to the lumen located within the inner layer, each of the at least four pathways positioned in a first concentric arrangement about and proximate to an outer circumference of the lumen and terminating proximate to at least one exterior surface of the outer layer for distributing fluid or gas from the first end of the flexible tip to the at least one outlet.

According to another embodiment of the present invention, a scope is disclosed which comprises:

a module having a least an illumination source, an image sensor, and a power source;

an elongated stylet having a first length with first and second ends and at least one centrally positioned inner lumen therein, the first end of the first length of the stylet proximate to the module, the first length of the elongated stylet having at least one pathway exterior to the at least one centrally positioned inner lumen;

the at least one centrally positioned inner lumen further comprising a first concentric arrangement, within the at least one inner lumen of the first length comprising at least one first fiber for illumination and at least one second fiber for receiving images;

the elongated stylet having a second length with first and second ends, the first end of the second length of the elongated stylet connected to the second end of the first length of the elongated stylet, the second end of the second length comprising a lens coupled to the at least one second fiber for receiving images, the second length of the elongated stylet having at least one centrally positioned inner lumen in communication with the at least one centrally positioned inner lumen of the first length of the elongated stylet;

the second length of the elongated stylet further comprising at least four pathways exterior to the at least one centrally positioned inner lumen of the second length of the elongated stylet, each of said at least four pathways positioned in a second concentric arrangement about and proximate to an outer circumference of the at least one centrally positioned inner lumen of the second length and terminating proximate to at least one exterior surface of the second length of the elongated stylet for distributing a gas, liquid, fluid or other substance supplied from the module through the at least one pathway of the first length and the at least four pathways of the second length.

According to yet another embodiment of the present invention, a scope is disclosed which comprises:

a module having a least an illumination source, an image sensor, and a power source:

an elongated stylet having a first length with first and second ends and at least one centrally positioned inner lumen therein, the first end of the first length of the stylet proximate to the module, the first length of the elongated stylet having at least one pathway exterior to the at least one centrally positioned inner lumen;

the at least one centrally positioned inner lumen further comprising a first concentric arrangement, within the at least one inner lumen of the first length comprising at least one first fiber for illumination and at least one second fiber for receiving images;

the elongated stylet having a second length with first and second ends, the first end of the second length of the elongated stylet connected to the second end of the first length of the elongated stylet, the second end of the second length comprising a lens coupled to the at least one second fiber for receiving images, the second length of the elongated stylet having at least one centrally positioned inner lumen in communication with the at least one centrally positioned inner lumen of the first length of the elongated stylet;

the second length of the elongated stylet further comprising at least four pathways exterior to the at least one centrally positioned inner lumen of the second length of the elongated stylet, each of said at least four pathways positioned in a second concentric arrangement about and proximate to an outer circumference of the at least one centrally positioned inner lumen of the second length and terminating proximate to at least one exterior surface of the second length of the elongated stylet for distributing a gas, liquid, fluid or other substance supplied from the module through the at least one pathway of the first length and the at least four pathways of the second length;

a display coupled to the module and in communication with the lens via the imaging fibers for viewing images proximate to the second end of the second length of the elongated stylet;

articulation wires extending longitudinally through the first and second lengths of the elongated stylet, the articulation wires coupled to a lever for manipulating the direction and/or orientation of the second end of the second length of the elongated stylet;

wherein the at least four pathways terminate about an outer circumference of the second length of the elongated stylet for ejecting a gas, liquid, fluid or other substance at a direction tangential to the longitudinal axis of the second length of the elongated stylet.

### BRIEF DESCRIPTION OF THE DRAWINGS

FIG. 1 is a perspective view of an endotracheal tube in accordance with the prior art;

FIG. 2 is a perspective view of an intubating scope in accordance with one embodiment of the present invention;

FIG. 3 is a perspective view of a connector used to fasten the intubating scope to the endotracheal tube;

FIG. 4 is a cross-sectional view of the stylet about axis A-A in FIG. 2;

FIG. 5 is a detailed perspective view of the flexible tip of the intubating scope of FIG. 2 according to one embodiment of the present invention;

FIG. 6 is a cross-sectional view of the flexible tip shown in FIG. 5;

FIG. 7 is a detailed perspective view of the flexible tip of the intubating scope of FIG. 5 in an alternate embodiment illustrating an optional side fluid port;

FIG. 8 is a detailed perspective view of the flexible tip of the intubating scope of FIG. 2 according to another alternate embodiment of the present invention;

FIG. 9 is a cross-sectional view of the flexible tip shown in FIG. 8;

FIG. 10 is a partial cross-sectional view of the flexible tip in an alternate embodiment;

FIG. 11 is another partial cross-sectional view of the flexible tip in an alternate embodiment;

FIG. 12 is another partial cross-sectional view of the flexible tip in an alternate embodiment; and

FIG. 13 is another partial cross-sectional view of the flexible tip in an alternate embodiment.

The drawings are not necessarily to scale, and may, in part, include exaggerated dimensions for clarity.

### DETAILED DESCRIPTION OF THE INVENTION

According to various embodiments, the present invention discloses a scope, such as a scope for performing intubation of a patient's airway, which is adapted to be used with an endotracheal tube during orotracheal intubation is provided that includes a module for manipulating the intubating scope. The module includes an illumination source, an image sensor, a power source, and a viewing member. Further, an elongated semi-malleable stylet including first and second ends and at least one inner lumen therein is connected to the module at the first end thereof. Additionally, a first end of a flexible tip is connected to a second end of the stylet. The flexible tip includes first and second ends, at least one inner lumen extending from the first end to the second end, and at least one pathway extending from the first end to the second end and spaced apart from the inner lumen.

Thus, the invention according to one embodiment provides an adjustable intubating scope to facilitate orotracheal insertion of an endotracheal tube (ETT) into a patient's larynx or trachea, and provide an unobstructed view in the area in front of a lens located on the flexible tip of the intubating scope. The lens may be connected to one or a variety of different media for displaying an image of the patient's larynx or trachea or other anatomy during intubation, and may further be displayed on one of a variety of display means, described in greater detail below. The intubating scope thus provides pathways for oxygen and other fluids to continually or intermittently clear and/or cleanse the area in front of the lens.

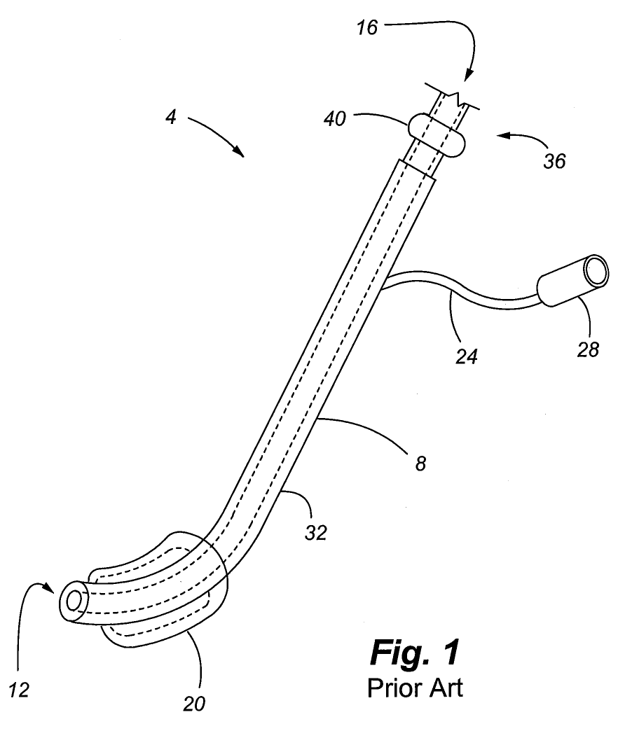

Referring to FIG. 1, a device according to one embodiment of the present disclosure is shown in a perspective view. The device in this embodiment comprises an ETT 4 for facilitating intubating a patient, and to ensure that the patient's airway is not closed off such that air is unable to reach the patient's lungs. The ETT 4 further comprises a shaft 8 having distal end 12 and proximal end 16 ends, a cuff 20 mounted near the distal end and a tube 24 mounted within and extending longitudinally through the shaft 8 and connected at a first end to the cuff 20. The ETT 4 further comprises a nozzle 28 mounted to the second end of the tube 24, a lumen 32 within the shaft 8 and a universal adaptor 36 having a lip piece 40.

Generally, the distal end 12 of the ETT 4 is inserted orotracheally into the patient, and the universal adaptor 36 is connected to a machine, such as a ventilator, which provides air to the patient's lungs via the lumen 32. While only a single lumen is shown, those of ordinary skill in the art will appreciate that multiple lumens can be provided to satisfy a user's specific requirements.

The cuff 20 is inflatable and is provided to form a seal with the wall of the trachea during intubation when inflated. Nozzle 28 connects the tube 24 to an inflation device (not shown). Accordingly, following insertion of the ETT 4 and when the cuff 20 is inflated with air, oxygen or other fluid, the exterior of the cuff 20 expands and contracts the interior of the patient's trachea so as to seal the trachea. Alternatively, the ETT 4 may have a pilot balloon (not shown) located under the nozzle 28 which can be manually squeezed to provide air to the cuff 20. Preferably the ETT 4 is molded to form a single continuous piece. Alternatively, the ETT 4 may be made from separate pieces of flexible plastic that are molded and connected into the shape shown in FIG. 1.

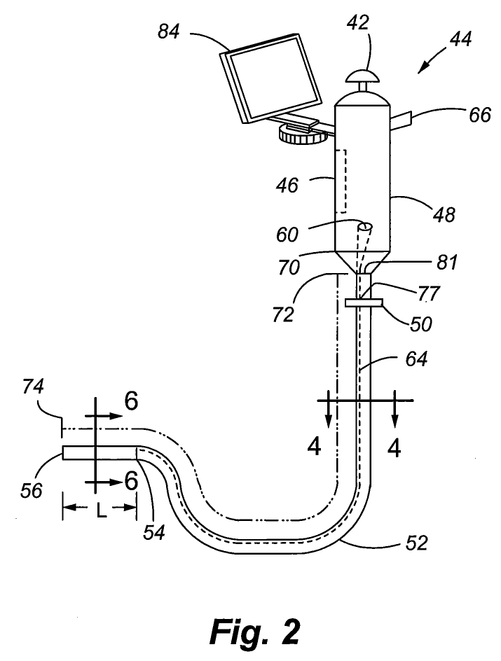

With reference to FIG. 2, a perspective view of an intubating scope 44 in accordance with one embodiment of the present invention is shown ideally for use in conjunction with an ETT 4, although the intubating scope 44 can be used in other applications as well. The intubating scope 44 generally includes a module 48 for manipulating the intubating scope 44, a stylet 52 for carrying the majority of the length of fiber-optic bundles, fluid pathways, etc., and a flexible tip 56 that can be manipulated by the module to allow an operator to view a patient's cavity.

The module 48 is preferably of an ergonomic shape to allow the module 48 to be easily grasped by an operator, and can include finger grips (not shown) to aid in retention by the operator. The outer shell of the module 48 can be constructed of any lightweight material such as aluminum, plastics, etc.

Housed within the module 48 is one end of a fiber-optic bundle 64, which may include illumination fibers and/or imaging fibers for allowing viewing of the patient's trachea, for example. An illumination source 77 provided proximate to a first end of the fiber-optic bundle 64 for providing illumination to a second end of the fiber-optic bundle 64 proximate to the flexible tip 56. The illumination source 77 may be a light emitting diode, for example, although ordinary artisans will appreciate that other light sources can be utilized. Additionally housed within the module is an image sensor 81, such as a charge-coupling device (CCD) chip, that is attached proximate to a first end of the fiber-optic bundle 64. The image sensor 81 receives photons that are received by a lens (not shown) attached to a second end of the fiber-optic bundle 64 located in the flexible tip 56 to provide images of the area viewed by the lens. It will be appreciated that other image sensor technology, such as a complementary metal-oxide-semiconductor (CMOS) chip, are also within the scope of the present invention.

A display screen 84, such as an LCD screen, is mounted to the module 48 and connected to the image sensor 81 via internal circuitry (not shown) to allow an operator to view the images received by the image sensor 81. The display screen 84 can be mounted in any way known in the art, and is preferably adjustable to provide a convenient viewing orientation regardless of the position of the module. In addition, the module can include an eyepiece 42 for allowing viewing of an image received by the image sensor 81. Additionally, an image-receiving port (not shown) may be provided in the module 48 and connected to the image sensor 81 via internal circuitry (not shown) to allow images to be transferred to an external device, such as a computer, for instance. Furthermore, a power source 46 provides power to the electrical components of the intubating scope 44. While the power source 46 is preferably at least one battery, the power source may also be an external power source, such as a standard 120-volt AC source that would connect to the module 48 via an electrical wire and plug. An on/off switch (not shown) may be provided to control supply of power from the power source 46 to the various electrical components.

Those of ordinary skill in the art will realize that while fiber-optic bundles 64 have been described, other transmission means such as electrical wiring or similar transmission cables are within the scope of the present invention. Further, it will be recognized that if electrical wires are used, the light source could be located in the flexible tip 56 rather than the module 48. Moreover, while the image sensor 81 is described as being located in the module 48, ordinary artisans will appreciate that the image sensor 81 could alternatively be located in the flexible tip 56 such that images would be transferred through the stylet 52 via cables to the LCD and/or image receiving port.

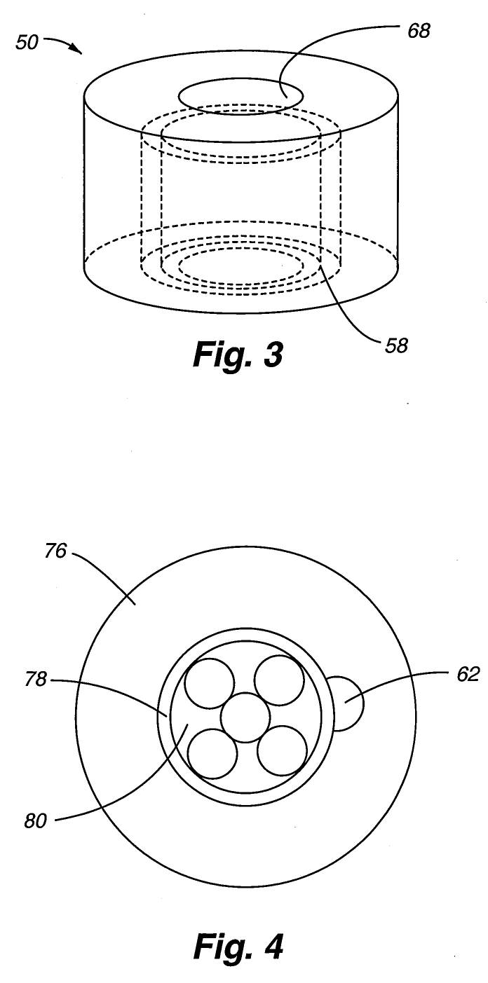

Slideable along the outer surface of the stylet 52 is a connector 50 used to mate adjacent the proximal end 16 of the ETT 4 to detachably fix the intubating scope to the ETT 4. With reference to FIG. 3, the connector 50 includes a central bore 58 that is friction fit around and slidable along the outer surface of the stylet 52. Additionally, the connector 50 includes cylindrical slot 68 adapted to frictionally receive the proximal end 16 of the ETT 4. Referring now to both FIGS. 3 and 2, in operation the connector 50 is first slid to a desired position along the outer surface of the stylet 52. Thereafter, the flexible tip 56 is inserted longitudinally into the ETT 4 until the proximal end 16 of the ETT 4 abuts the connector 50. Finally, the proximal end 16 is inserted into the cylindrical slot 68 of the connector 50 to detachably fix the intubating scope 44 to the ETT 4. The connector 50 is constructed of any of various plastics, metals, etc. Alternatively, rather than utilizing a friction-fit connection, the connector 50 may utilize a fastener, such as a set-screw (not shown), that engages the proximal end 16 of the ETT 4. Alternatively, the fastener may be a member having a cam-shaft surface that is rotated to engage the underside of the lip piece 40.

In addition to the illumination and imaging fibers connected to the module 48, pathways are provided for transferring various fluids from the module 48 through the stylet 52 to the flexible tip 56. Referring now to FIGS. 2 and 4, a first end of each pathway is an inlet 60 or other valve known in the art (i.e. threaded connection) for introducing the fluids into a pathway 62. A second distal end of pathway 62 is connected to a port in the flexible tip 56 for allowing exit of the fluids introduced therein. Pathway 62 can be of various constructions, including tubing, conduits, ducts, etc. For instance, one or more pathways 62 can be for receiving a non-toxic solution such as lidocaine or saline for cleansing of the lens at the second distal end of the fiber-optic bundle 64 due to oropharyngeal blood or other secretions or debris in front of or on the lens as will be described below. Additionally, a pathway may be formed within the stylet 52 that runs from an inlet 60 in the module 48 to an outlet in the flexible tip 56. The pathway may receive oxygen for removing secretions and/or displacing soft tissue atraumatically, from the area in front of the lens as described in greater detail below. While the pathways are illustrated as being mounted within the stylet 52, it is contemplated that one or more of the pathways could be formed of a separate structure, such as a tubing structure, and run along side the stylet 52 to the flexible tip 56 and fixed to the stylet 52 via clips, adhesive, etc.

Continuing with reference to FIG. 2, the module 48 additionally includes a lever 66 to manipulate the flexible tip 56 in an up and down direction in one plane. More specifically, at least two articulation wires (not shown) are connected to the lever 66, run through the stylet 52, and are mounted to the flexible tip 56. Preferably, the articulation wires are positioned opposite each other and extend longitudinally to the flexible tip 56 so as to impart opposing flexible forces which provide the up and down motion of the flexible tip 56 upon movement of the lever in an up or down motion. However, other means may be employed to manipulate the flexible tip 56 in an up and down motion. For instance, if the flexible tip 56 is biased in either the up or down direction, then only a single wire could be utilized to overcome the bias and flex the flexible tip 56 in the opposite direction. By rotating the module, and thereby the flexible tip 56, a user may change the plan in which the flexible tip 56 is moved relative to the axis of movement With continued reference to FIG. 2, a junction 70 is located on the module 48 and proximate to the stylet 52 for receiving the fiber optic bundles 64, pathway 62, articulation wires, etc. The junction 70 is preferably rigid to allow the operator to manipulate the majority of the stylet 52 by manipulating the module 48, although a flexible junction is also contemplated as being within the scope of the present invention. For instance, a ball and socket joint with a threaded locking pin would be useful for a difficult situation when the operator needs to change the angle between the module 48 and stylet 52, and rigidly maintain that angle thereafter.

The stylet 52 has first end 72 and second end 74, and is preferably constructed of a semi-malleable material, and has an internal geometry for receiving the fiber-optic bundles 64, pathway 62, articulation wires, etc. The material can be aluminum or other flexible metal, such as medical-grade plastic, etc. As shown in FIG. 2, the stylet 52 has been formed into a substantially J-shaped configuration for simulating the anatomical curvature made between the tongue and soft palate when the patient is in the supine position. However, ordinary artisans will realize that numerous other shapes can be formed to accommodate individual patients. In addition, the stylet 52 and flexible tip 56 may be covered with a soft clear coating such as a thermoplastic material to protect the stylet 52 and flexible tip 56 during sterilization and from any water-soluble lubricants used to facilitate easy insertion and removal of the stylet 52 and flexible tip 56 into and out of the ETT 4, as well as preventing trauma to the trachea caused by contact with the flexible tip 56 of the stylet 52.

Referring now to FIG. 4, a cross-sectional view of the stylet 52 of FIG. 2 is shown. More specifically, the stylet 52 according to this embodiment includes the inner layer 78 which forms an inner lumen 80, and further includes an outer layer 76, which has formed therein a pathway 62 for transporting oxygen or other gases or fluids from the module 48 to the flexible tip 56. Preferably, the inner layer 78 and outer layer 76 of the stylet 52 are manufactured by extruding molten polymer. The pathway 62 can be formed during the manufacturing process or can be carved out of the outer layer 76 thereafter. However, it will be appreciated that tubing could be placed into the pathway 62 or otherwise formed in the outer layer 76 for carrying the oxygen or other gases or fluids from the module 48 to the flexible tip 56.

The pathway 62 is connected at a first end to an inlet 60 in the module 48 and at a second end to a second end 54 of the stylet 52. After forming, the inner layer 78 and outer layer 76 are laminated or adhered together, rolled and cut to form the completed stylet 52. However, those of ordinary skill in the art will appreciate that various other manufacturing methods can be used, such as molding, welding, extruding, etc. Additionally, other materials could be used such as aluminum, copper, composites, etc. Moreover, although the fiber-optic bundles 64, pathway 62, inner layer 78 and outer layer 76 are in the particular orientation as shown in FIG. 4, it will be recognized that other orientation of the bundles, pathways and wires is contemplated as being within the scope of the present invention.

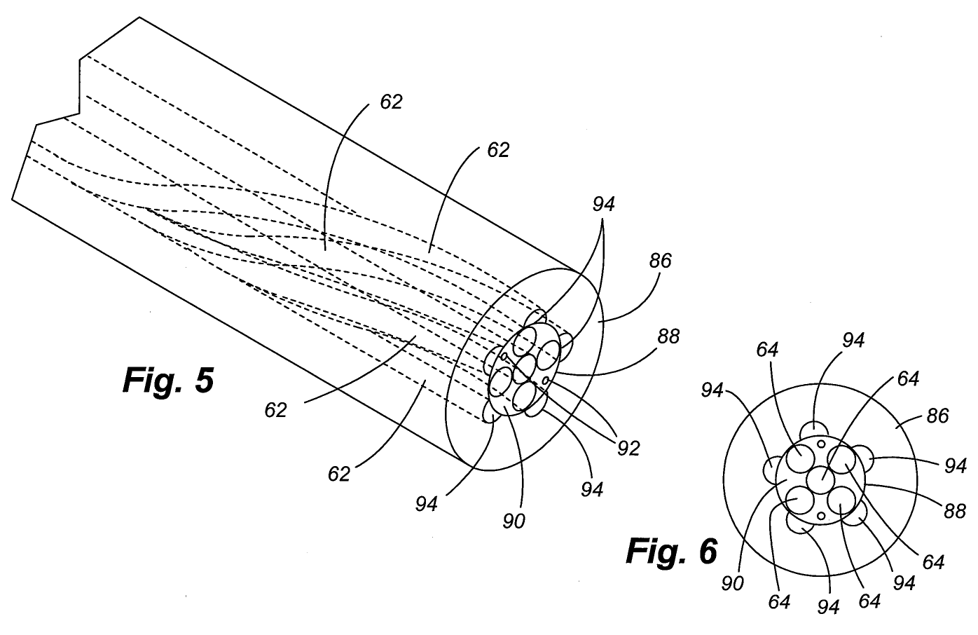

Referring now to FIG. 5, a detailed perspective view of the flexible tip 56 shown in FIG. 2 according to one alternative embodiment illustrated. The flexible tip 56 is preferably constructed to be more flexible than the stylet 52 so as to allow the flexible tip 56 to be manipulated by the lever 66 while the stylet 52 maintains its shape during orotracheal insertion. The flexible tip 56 can be constructed of soft metals, thermoplastics, medical-grade plastics, etc. Similar to the stylet 52, the flexible tip 56 includes an outer layer 86, an inner layer 88 and an inner lumen 80. Additionally, the flexible tip 56 includes first end 72 and second end 74 ends and an outer layer 86. The first end 72 of the flexible tip 56 is connected to the second end 74 of the stylet 52 by any means known in the art, such as adhesive, fusing, welding, etc. Additionally, a metal ring (not shown) can be provided at the junction of the stylet 52 and the flexible tip 56 to prevent against rupture of the flexible tip 56 from the stylet 52. Preferably, the diameters of the outer layer 86, inner layer 88 and inner lumen 80 are equivalent to those of the stylet 52 so as to provide continuity throughout the length of the stylet 52.

Further, the inner lumen 80 of the stylet 52 leads directly into the inner lumen 80 of the flexible tip 56 such that both of the image sensor 81, define a single continuous lumen extending from the module 48 to the end of the flexible tip 56. Thus, the fiber-optic bundles 64, pathways 62 and articulation wires 92 extend from the stylet 52 directly to the second end 74 of the flexible tip 56. The ends of the fiber-optic bundles 64 include transparent caps, for example, to project light from the light source into the area in front of the second end of the flexible tip 56. Moreover, one end of the fiber-optic bundle 64 includes a lens for receiving images illuminated by the light source and sending the images received to the image sensor via the fiber-optic bundle 64. Additionally, the ends of the pathways 62 include outlet ports 94 for ejecting fluids or gases sent down the pathways 62 to the area about and/or surrounding the lens and/or transparent cap. While the ends of the fiber-optic bundles 64, pathways 62 and articulation wires 92 are shown to be fixed right at the first end 72 of the flexible tip 56, ordinary artisans will appreciate that the flexible tip 56 or fiber-optic bundles 64, pathways 62 and articulation wires 92 can be constructed such that the fiber-optic bundles 64, pathways 62 and articulation wires 92 end either before or after the first end 72 of the flexible tip 56. Further, the ends of the fiber-optic bundles 64, pathways 62 and articulation wires 92 can be fixed to or near the first end 72 of the flexible tip 56 in any means known in the art such as by adhesives, bonding, compression of the inner and outer layers, etc. Additionally, an end cap that fits over the fiber-optic bundles 64, pathways 62 and articulation wires 92 that includes bores for receiving the fiber-optic bundles 64, pathways 62 and articulation wires 92 could be utilized for fixing the fiber-optic bundles 64, pathways 62 and articulation wires 92 to the flexible tip 56. The end cap could be friction fitted or otherwise secured into the inner lumen 80 of the flexible tip 56 from the second end 74 of the flexible tip 56.

With continued reference to FIGS. 5 and 6, the outer layer 86 of the flexible tip 56 also includes a number of pathways 62 extending from the first end 72 of the flexible tip 56 to the second end 74 of the flexible tip 56. The pathways 62 can be formed in the outer layer 86 as part of a molding process. Alternatively, the pathways 62 can be formed in the outer layer 86 after manufacturing of the outer layer 86 and before the outer layer 86 is laminated or otherwise bonded to the inner layer 88. Each of the pathways 62 meet proximate to the second end 74 of the flexible tip 56 and radiate towards the first end 72 of the flexible tip 56 spaced around the inner lumen 80.

In operation, the pathways 62 receive oxygen or other fluids from the pathways 62 of the stylet 52 and distribute the oxygen or other fluids to the pathways 62. Thereafter, the fluids exit the pathways 62 proximate to the first end 72 of the flexible tip 56. As the oxygen or fluids exit the pathways 62, they clear debris, secretions, soft tissue, etc. from the area in front of the lens and/or transparent cap. Because the pathways 62 completely surround the lens and transparent cap, a “clean zone” is formed in front of the lens and illumination caps thus allowing the lens and image sensor to receive an unobstructed view of a particular area of the patient. While only five pathways 62 have been shown, ordinary artisans will realize that more or fewer pathways 62 can be provided depending on a particular application of the intubating scope 44. Further, the outlet ports 94 of the pathways 62 may comprise any number of shapes, including but not limited to semi-circular, circular, rectangular, etc., and may exit the second end 74 of the flexible tip 56 at any number of directions to provide fluid flow in a desired direction.

Preferably, the oxygen or fluid flow is directed perpendicularly or outward with respect to the second end 74 of the flexible tip 56. Also, while pathways 62 have been formed directly in the outer layer 86 of the flexible tip 56, it is contemplated that tubing could be mounted in the outer layer 86 of the flexible tip 56 to transport oxygen or other fluids through the stylet 52 to the area in front of and surrounding the lens and transparent cap at the second end 74 of the flexible tip 56. To further create the clean zone, a non-toxic solution such as lidocaine or saline can be injected into the inlets 60 in the module 48. The solution will then travel down the pathways 62 and exit from the outlet ports 94 located near the first end 72 of the flexible tip 56 thus cleaning the area on and around the lens and transparent cap.

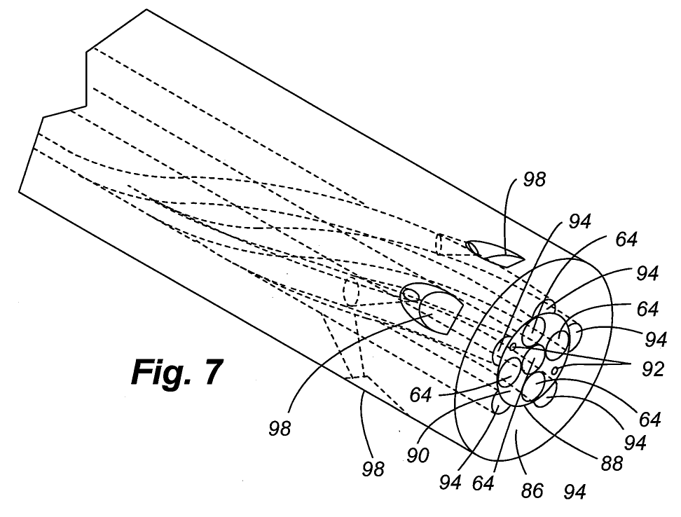

Referring to FIG. 7, a variation of the flexible tip of FIG. 5 is shown including at least one side fluid port 98 for transporting fluids to the outer layer 86 of the flexible tip 56 near the second end 74 of the flexible tip 56. While only three side fluid ports 98 are illustrated for clarity, it is contemplated that each of the pathways 62 may have corresponding side fluid ports 98. Similar to the pathways 62, each side fluid port 98 may be formed in the outer layer 86 during the molding process or formed thereafter. Additionally, while the side fluid port 98 is shown branching from one of the pathways 62, it is contemplated that the side fluid port 98 could begin proximate the second end 54 of the stylet 52 and then terminate at the outer layer 86 of the flexible tip 56 near the first end 72 of the flexible tip 56.

Thus, in operation, oxygen or other fluids traveling through the stylet 52 and into the flexible tip 56 will be distributed through the pathways 62 and also the side fluid ports 98. As a result, while the oxygen or other fluid from the pathways 62 clear debris from the area in front of the lens and transparent cap, the oxygen or other fluid from the side fluid ports 98 will clear debris approaching the lens and transparent cap from the lateral side of the second end 74 of the flexible tip 56, thus enhancing the clean zone in front of the second end 74 of the flexible tip 56, and allowing a greater degree of movement of the flexible tip in the cavity or orifice of the patent.

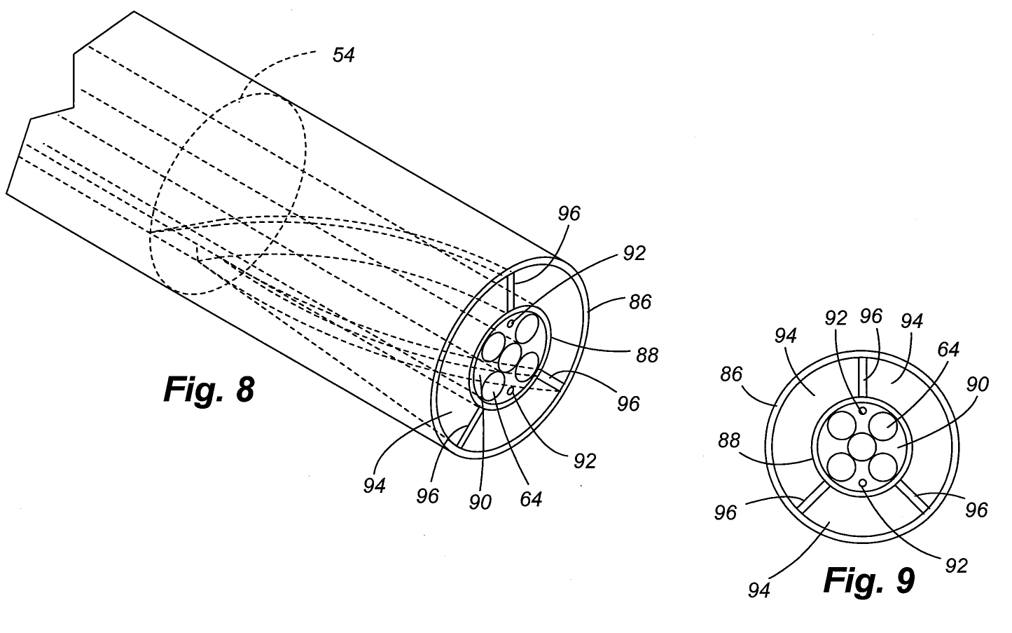

With reference to FIGS. 8 and 9, another embodiment of the flexible tip 56 of the present invention is shown. The flexible tip 56 includes an outer layer 86, an inner layer 88, an inner lumen 80, a first end 72, and a second end 74. Additionally, the flexible tip 56 is designed to provide a gap or space between the inner 88 and outer 86 layers for fluid flow as will be described hereinafter.

The pathway 62 of the stylet 52 carries oxygen or other fluid into the outlet ports 94 at the first end 72 of the flexible tip 56. Situated in the outlet ports 94 and on each side of the pathway 62 as it enters the flexible tip 56 are elongated ribs 96 for directing the oxygen or other fluid from the receiving area to the second end 74 of the flexible tip 56 and for providing structural support between the inner and outer layers. Each of the elongated ribs 96 provide lateral support to the outer layer 86, and further provide direction for distributing the oxygen or other fluid through the outlet ports 94. Ordinary artisans will realize that more than one supporting elongated rib 96 may be provided for additional lateral support thereto.

Because of the symmetrical orientation of the elongated ribs 96, substantially equal quantities of oxygen or other fluid will be delivered to each of the outlet ports 94. With particular reference to the second end 74 of the flexible tip 56, it will be recognized that the outlet ports 94 provide for the flow of oxygen or other fluid around substantially about the entire circumference of the inner lumen 80. Thus, a clean zone is created in the area in front of the second end 74 of the flexible tip 56 for clearing debris, secretions and soft tissue away from the lens and transparent cap. Those of ordinary skill in the art will appreciate that additional elongated ribs 96 with varying orientations and/or directions within the flexible tip 56 may be further provided to direct fluid flow to a particular location. Further, it is contemplated that the previously described side fluid ports 98 may be incorporated as shown in FIG. 7 to provide fluid flow to the lateral surface of the flexible tip if desired.

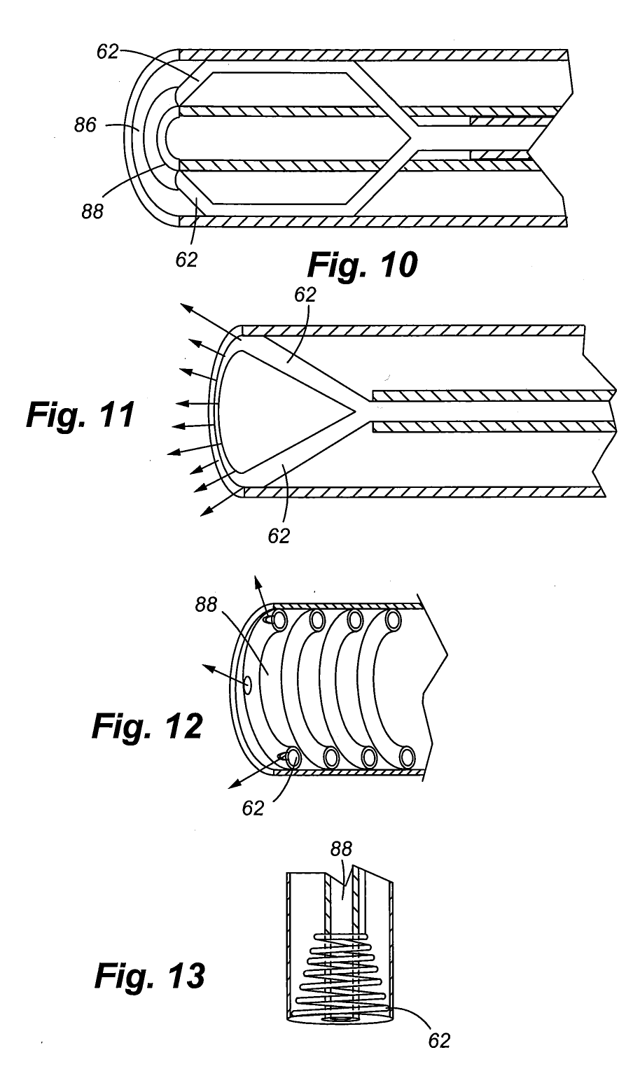

Referring now to FIGS. 10A-C and 11, a flexible tip according to various alternative embodiments are shown. As shown in FIG. 10A, pathways 62 running through the flexible tip are oriented in a direction to allow gas or liquid exiting the outlets to be forced at least partially across the surface of the lens and/or inner lumen. As shown in FIG. 10B, the pathways 62 are oriented to direct and distribute gas or liquid in a somewhat radial orientation about the second end of the flexible tip, thereby providing a pushing force in both the longitudinal direction and the lateral direction with respect to the flexible tip and stylet. As shown in FIG. 10C, the pathways are oriented to distribute gas or liquid in a helical or spiral flow-pattern, thereby providing both longitudinal and lateral force to any surrounding tissue or debris. It is to be expressly understood that while these alternate embodiments are depicted each with one type of pathway, combinations of these various alternative embodiments may be combined to provide the optimal force required for the particular application, or to provide both clearing of tissue and/or debris along with periodic cleansing of the lens. Although the drawings depict the pathways to be consistent with respect to the diameter of the pathways, in other alternative embodiments the pathways may change from a larger diameter to a smaller diameter as the pathways approach the second end of the flexible tip, thereby increasing the flow-rate of the gas or liquid therein. In yet another alternative embodiment, the pathways may change from a smaller diameter to a larger diameter as they approach the second end of the flexible tip.

Referring now to FIG. 11, another alternative embodiment of the present disclosure is shown. In this embodiment, a single pathway 62 is shown extending from the first end of the flexible tip adjacent the inner lumen, to the second end of the flexible tip adjacent the outer surface of the flexible tip. This orientation may be desirable for providing a helical or spiral flow patterns about the surface of the lens and the second end of the flexible tip. Although only a single pathway 62 is depicted in FIG. 11, additional pathways may be incorporated with similar or dissimilar orientations without deviating from the present inventive concepts described herein.

The outer diameter of the stylet 52 and flexible tip 56 of the intubating scope 44 may be of varying degrees to satisfy a particular application of the intubating scope 44. For instance, if the intubating scope 44 is used in conjunction with an ETT 4, the outer diameter of the intubating scope 44 must be smaller than the inner diameter of the lumen 32 of the ETT 4. However, if the intubating scope 44 is used independent of the ETT 4, then the outer diameter of the intubating scope 44 can be any size appropriate to be placed into the cavity (i.e. larynx) of a particular patient. An initial range is contemplated to be 4.0 mm-6.5 mm. More specifically, a more preferred range is contemplated to be 4.5 mm-6.0 mm. Finally, the preferred range is 5.5 mm-6.0 mm. These ranges may vary for different applications other than insertion via the larynx, depending on the cavity or orifice of the patient.

Further, the length of the stylet 52 and flexible tip 56 can be of almost any dimension to suit a particular application of the intubating scope 44. In general, the cope may be larger for a larger patient, or smaller for a smaller patient. For instance, an average adult male will likely require a longer stylet 52 and flexible tip 56 than will an infant child for an effective orotracheal intubation. However, if the intubating scope 44 is to be used in conjunction with an ETT 4, the length of the stylet 52 and flexible tip 56 should not be much shorter than the length of the ETT 4 to allow the lens to effectively receive images during intubation. An initial range of the length of the stylet and flexible tip is 30.0 cm-53.0 cm. An intermediate range is contemplated as 35.0 cm-45.0 cm. Finally, a preferred range is from 38.0 cm-40.0 cm. Further, while the above ranges include both the stylet 52 and the flexible tip 56, the flexible tip 56 alone is preferably from 3.5 cm-4.5 cm in length, as shown in FIG. 2 as length L, although may be of shorter or longer lengths to accommodate the specific application. For example, certain borescope applications may require a length in excess of 53 cm, including up to 100 cm.

Additionally, the outer and inner diameters of the ETT 4 can be of almost any dimension to accommodate a particular patient. For instance, the inner diameter of the lumen 32 of the ETT 4 must be larger than the outer diameter of the stylet 52 and flexible tip 56 of the intubating scope 44. Additionally, the outer diameter of the ETT 4 might need to be larger or smaller depending upon the size of the airway in the patient. According to one embodiment of the present invention, the inner diameter measures within the range of 4.5 mm-10.0 mm. More preferably, the range of the inner diameter is within 5.5 mm-9.0 mm. Finally, the preferred range of the inner diameter is contemplated to be 6.5 mm-7.5 mm. The range of the outer diameter is contemplated to be 6.5 mm-15.0 mm. More preferably, the range of the outer diameter is within 8.0 mm-12.0 mm. Finally, the preferred range of the outer diameter is contemplated to be 9.5 mm-10.5 mm.

Further, the length of the ETT 4 can be of almost any dimension to allow effective oxygen flow from the mouth to the lungs of a particular patient. For instance, an average adult male will likely require a longer ETT 4 than will an infant child for an effective orotracheal intubation. An initial range of the length of the ETT 4 is 25.0 cm-45.0 cm. An intermediate range is contemplated as between 28.0 cm-38.0 cm. Finally, a preferred range is from 31.0 cm-33.0 cm.

The preferred oxygen flow rate into the inlet 60 of the stylet 52 is 5.0 L/min-10.0 L/min to allow for effective oxygen flow from the outlets near the second end 74 of the flexible tip 56. However, those of ordinary skill in the art will appreciate other flow rates outside of this stated range may be appropriate in specific situations.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together. Although certain combinations or subcombinations have been described in discrete paragraphs, it is to be expressly understood that any multiple combination of the components may be provided as reflected in the following claims.

The present invention, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The present disclosure, although relying on the description of a scope for intubating, is expressly intended to include scopes for other applications as well. For example, a Videoscope or Video Borescope is another type of scope that may include a small CCD chip embedded into the tip of the scope. The video image is relayed from the distal tip and focusable lens assembly back to the display via internal wiring. Alternatively, a traditional Borescopes relies on optical relay components to transfer the image from the tip to an eyepiece, and Fiberscopes use coherent image fiberoptics to relay the image to an eyepiece. These systems normally provide the ability to capture the images and to record those images via either live video or still photos.

What is claimed is:

1. A flexible tip for use with a scope, the flexible tip comprising:
a first end and a second end;
an outer layer and an inner layer extending longitudinally from the first end to the second end;
a lumen located within the inner layer and extending longitudinally from the first end to the second end;
the flexible tip further comprising at least four pathways exterior to the lumen located within the inner layer, each of the at least four pathways positioned in a first concentric arrangement about and proximate to an outer circumference of the lumen and terminating proximate to at least one exterior surface of the outer layer for distributing fluid or gas from the first end of the flexible tip to the at least one outlet.

2. The flexible tip of claim 1, further comprising a scope, a module for manipulating the scope, the module including an illumination source, an image sensor, a power source, and an elongated stylet including first and second ends and at least one inner lumen therein, the first end of the stylet connected to the module and the second end of the stylet connected to the first end of the flexible tip.

3. The flexible tip of claim 2, further comprising articulation wires extending longitudinally through the flexible tip and accessible from the module to manipulate the direction of the flexible tip with respect to the longitudinal axis of the flexible tip.

4. The flexible tip of claim 1, wherein the at least four pathways are oriented to distribute fluid or gas in both a direction substantially parallel to the longitudinal axis of the flexible tip and substantially tangential to the longitudinal axis of the flexible tip.

5. The flexible tip of claim 1, wherein the at least four pathways are positioned in a substantially helical or spiral direction about the lumen and terminate at the second end and at an outer circumference of the outer layer of the flexible tip.

6. The flexible tip of claim 1, wherein the at least four pathways terminate about an outer circumference of the flexible tip for ejecting a gas, liquid, fluid or other substance at a direction substantially tangential to the longitudinal axis of the flexible stylet.

7. The flexible tip of claim 2, further comprising a display for viewing the images via the lens and the at least one second fiber for receiving images.

8. The flexible tip of claim 7, wherein the display is selected from the group consisting of a LCD monitor, a plasma monitor, a thin film display monitor, and a high definition monitor.

9. The flexible tip of claim 2 wherein the scope is selected from the group consisting of a borescope, a colonoscope, an intubation scope, a neurosurgical scope, and a fiberscope.

10. A scope comprising:
a module having a least an illumination source, an image sensor, and a power source:
an elongated stylet having a first length with first and second ends and at least one centrally positioned inner lumen therein, the first end of the first length of the stylet proximate to the module, the first length of the elongated stylet having at least one pathway exterior to the at least one centrally positioned inner lumen;
the at least one centrally positioned inner lumen further comprising a first concentric arrangement, within the at least one inner lumen of the first length comprising at least one first fiber for illumination and at least one second fiber for receiving images;
the elongated stylet having a second length with first and second ends, the first end of the second length of the elongated stylet connected to the second end of the first length of the elongated stylet, the second end of the second length comprising a lens coupled to the at least one second fiber for receiving images, the second length of the elongated stylet having at least one centrally positioned inner lumen in communication with the at least one centrally positioned inner lumen of the first length of the elongated stylet;
the second length of the elongated stylet further comprising at least four pathways exterior to the at least one centrally positioned inner lumen of the second length of the elongated stylet, each of said at least four pathways positioned in a second concentric arrangement about and proximate to an outer circumference of the at least one centrally positioned inner lumen of the second length and terminating proximate to at least one exterior surface of the second length of the elongated stylet for distributing a gas, liquid, fluid or other substance supplied from the module through the at least one pathway of the first length and the at least four pathways of the second length.

11. The scope of claim 10, further comprising a display coupled to the module and in communication with the lens via the imaging fibers for viewing images proximate to the second end of the second length of the elongated stylet.

12. The scope of claim 10, further comprising articulation wires extending longitudinally through the first and second lengths of the elongated stylet, the articulation wires coupled to a lever for manipulating the direction and/or orientation of the second end of the second length of the elongated stylet.

13. The scope of claim 10 wherein the at least four pathways terminate about an outer circumference of the second length of the elongated stylet for ejecting a gas, liquid, fluid or other substance at a direction tangential to the longitudinal axis of the second length of the elongated stylet.

14. A scope comprising:
a module having a least an illumination source, an image sensor, and a power source:
an elongated stylet having a first length with first and second ends and at least one centrally positioned inner lumen therein, the first end of the first length of the stylet proximate to the module, the first length of the elongated stylet having at least one pathway exterior to the at least one centrally positioned inner lumen;
the at least one centrally positioned inner lumen further comprising a first concentric arrangement, within the at least one inner lumen of the first length comprising at least one first fiber for illumination and at least one second fiber for receiving images;
the elongated stylet having a second length with first and second ends, the first end of the second length of the elongated stylet connected to the second end of the first length of the elongated stylet, the second end of the second length comprising a lens coupled to the at least one second fiber for receiving images, the second length of the elongated stylet having at least one centrally positioned inner lumen in communication with the at least one centrally positioned inner lumen of the first length of the elongated stylet;
the second length of the elongated stylet further comprising at least four pathways exterior to the at least one centrally positioned inner lumen of the second length of the elongated stylet, each of said at least four pathways positioned in a second concentric arrangement about and proximate to an outer circumference of the at least one centrally positioned inner lumen of the second length and terminating proximate to at least one exterior surface of the second length of the elongated stylet for distributing a gas, liquid, fluid or other substance supplied from the module through the at least one pathway of the first length and the at least four pathways of the second length;
a display coupled to the module and in communication with the lens via the imaging fibers for viewing images proximate to the second end of the second length of the elongated stylet;
articulation wires extending longitudinally through the first and second lengths of the elongated stylet, the articulation wires coupled to a lever for manipulating the direction and/or orientation of the second end of the second length of the elongated stylet;
wherein the at least four pathways terminate about an outer circumference of the second length of the elongated stylet for ejecting a gas, liquid, fluid or other substance at a direction tangential to the longitudinal axis of the second length of the elongated stylet.

15. The scope of claim 14 wherein the scope is adapted to be used with an endotracheal tube during orotracheal intubation.

16. The scope of claim 14 wherein the scope is selected from the group consisting of a borescope, a colono scope, an intubation scope, a neuro surgical scope, and a fiberscope.
